(19) United States
(12) Patent Application Publication
(10) Pub. No.: US 2026/0083336 A1
(43) Pub. Date: Mar. 26, 2026

(54) INFORMATION PROCESSING DEVICE, MONITORING DEVICE, AND NON-TRANSITORY COMPUTER-READABLE MEDIUM

(71) Applicant: NIHON KOHDEN CORPORATION, Tokyo (JP)

(72) Inventor: Tetsuri ARIYAMA, Saitama (JP)

(73) Assignee: NIHON KOHDEN CORPORATION, Tokyo (JP)

(21) Appl. No.: 19/326,176

(22) Filed: Sep. 11, 2025

(30) Foreign Application Priority Data
Sep. 20, 2024 (JP) 2024-163953

(51) Int. Cl. A61B 5/0205; A61B 5/00; A61B 5/02; A61B 5/024; A61B 5/08; G16H 40/67

(52) U.S. Cl. CPC A61B 5/0205; A61B 5/7235; G16H 40/67; A61B 5/02007; A61B 5/024; A61B 5/0816

(57) ABSTRACT

An interface receives a signal corresponding to a waveform of physiological information involving cyclic changes from a sensor attached to a subject. A processor causes, in response to the signal, an output device to output information corresponding to an estimated value of a respiration rate of the subject. The processor extracts multiple feature quantities corresponding to respiration of the subject from the signal. The processor applies frequency analysis processing with respect to each of the feature quantities to acquire multiple preliminary estimation values of the respiration rate. The processor excludes one of the preliminary estimation values in response to state information corresponding to a state of the subject. The processor determines the estimated value based on at least one of the preliminary estimation values as remained.

### CROSS-REFERENCE TO RELATED APPLICATION

The present application is based on Japanese Patent Application No. 2024-163953 filed on Sep. 20, 2024, the entire contents of which are incorporated herein by reference.

### BACKGROUND

The presently disclosed subject matter relates to an information processing device configured to process physiological information of a subject. The present disclosure also relates to a monitoring device configured to monitor physiological information of a subject. The presently disclosed subject matter also relates to a non-transitory computer-readable medium having stored a computer program adapted to be executed by a processor installed in the information processing device.

It is well-known a technique in which a waveform of pulse waves that is an example of a waveform of physiological information involving cyclic changes is acquired from a subject to estimate a respiration rate of the subject based on the waveform. Japanese Patent No. 3627243B discloses that body motions of a subject is detected with an acceleration sensor to improve estimation accuracy of the respiration rate. Japanese Patent No. 6355152B discloses that the number of sensors for acquiring the waveform of the pulse waves to improve estimation accuracy of the respiration rate.

### SUMMARY

It is required to suppress deterioration of the estimation accuracy of the respiratory rate in accordance with a state of the subject without increasing the number of sensors.

An illustrative aspect of the presently disclosed subject matter may provide an information processing device configured to process physiological information of a subject, comprising:
an interface configured to receive a signal corresponding to a waveform of physiological information involving cyclic changes from a sensor attached to the subject; and
a processor configured to cause, in response to the signal, an output device to output information corresponding to an estimated value of a respiration rate of the subject,
wherein the processor is configured to:
   extract multiple feature quantities corresponding to respiration of the subject from the signal;
   apply frequency analysis processing with respect to each of the feature quantities to acquire multiple preliminary estimation values of the respiration rate;
   exclude one of the preliminary estimation values in response to state information corresponding to a state of the subject; and
   determine the estimated value based on at least one of the preliminary estimation values as remained.

An illustrative aspect of the presently disclosed subject matter may provide a monitoring device configured to monitor physiological information of a subject, comprising:
an interface configured to receive a signal corresponding to a waveform of physiological information involving cyclic changes from a sensor attached to the subject;
an output device; and
a processor configured to cause, in response to the signal, the output device to output information corresponding to an estimated value of a respiration rate of the subject,
wherein the processor is configured to:
   extract multiple feature quantities corresponding to respiration of the subject from the signal;
   apply frequency analysis processing with respect to each of the feature quantities to acquire multiple preliminary estimation values of the respiration rate;
   exclude one of the preliminary estimation values in response to state information corresponding to a state of the subject; and
   determine the estimated value based on at least one of the preliminary estimation values as remained.

An illustrative aspect of the presently disclosed subject matter may provide a non-transitory computer-readable medium having stored a computer program adapted to be executed by a processor installed in an information processing device configured to process physiological information of a subject, the computer program being configured to, when executed, cause the information processing device to:
receive a signal corresponding to a waveform of physiological information involving cyclic changes from a sensor attached to the subject;
extract multiple feature quantities corresponding to respiration of the subject from the signal;
apply frequency analysis processing with respect to each of the feature quantities to acquire multiple preliminary estimation values of a respiration rate of the subject;
exclude one of the preliminary estimation values in response to state information corresponding to a state of the subject;
determine an estimated value based on at least one of the preliminary estimation values as remained; and
cause an output device to output information corresponding to the estimated value.

According to the configuration of each of the above illustrative aspects, since the processing for excluding the preliminary estimation value that deteriorates the estimation accuracy of the respiration rate based on the state of the subject is executed by the processor installed in the information processing device, it is possible to suppress the deterioration in the estimation accuracy of the respiration rate in accordance with the state of the subject without increasing the number of sensors.

### DESCRIPTION OF THE DRAWINGS

FIG. 1 illustrates a functional configuration of a monitoring device according to an exemplary embodiment.

FIG. 2 illustrates a flow of processing executed by a processor of FIG. 1.

FIG. 3 illustrates a baseline fluctuation of a waveform signal of FIG. 1.

FIG. 4 illustrates an amplitude fluctuation of the waveform signal of FIG. 1.

FIG. 5 illustrates a frequency fluctuation of the waveform signal of FIG. 1.

FIG. 6 illustrates an exemplary flow of selection processing of FIG. 2.

FIG. 7 illustrates another exemplary flow of the selection processing of FIG. 2.

FIG. 8 illustrates a waveform of pulse waves that is acquired from a subject whose vascular age is relatively low.

FIG. 9 illustrates a waveform of pulse waves that is acquired from a subject whose vascular age is relatively high.

### DESCRIPTION OF EMBODIMENTS

Exemplary embodiments will be described in detail with reference to the accompanying drawings.

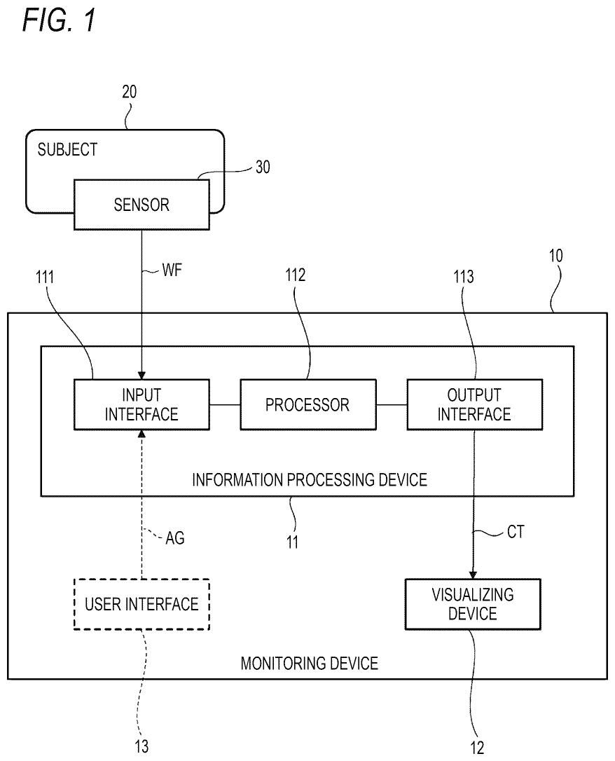

FIG. 1 illustrates a functional configuration of a monitoring device 10 according to an exemplary embodiment. The monitoring device 10 is a device configured to monitor physiological information of the subject 20. The monitoring device 10 includes an information processing device 11 and a visualizing device 12.

The information processing device 11 is a device configured to process physiological information of a subject 20. Specifically, the information processing device 11 is configured to acquire information corresponding to an estimated value of a respiratory rate of the subject 20 based on a signal corresponding to a waveform of pulse waves of the subject 20. The pulse waves is an example of physiological information involving cyclic changes. As used herein, the term “waveform”means a time series of values of specific physiological information.

The visualizing device 12 is configured to visualize information corresponding to an estimated value of the respiratory rate of the subject 20. As an example, the visualizing device 12 may be a display configured to display the information, or a projector configured to project the information. As another example, the visualizing device 12 may be a printer configured to print the information on a medium. The visualizing device 12 is an example of an output device.

The information processing device 11 includes an input interface 111. The input interface 111 is configured as a hardware interface that receives a waveform signal WF corresponding to the waveform of the pulse waves from a sensor 30 attached to the subject 20. The waveform signal WF may be an analog signal or a digital signal in accordance with the specification of the sensor 30.

Examples of the sensor 30 for acquiring the pulse waves include a photoelectric sensor including a light emitting element and a light detecting element. The light emitted from the light emitting element passes through a living tissue of the subject 20, and is incident on the light detecting element. In accordance with pulsations of the subject 20, the intensity of the incident light on the light detecting element is changed. Accordingly, changes over time of the incident light intensity may correspond to a waveform of the pulse waves.

In a case where the waveform signal WF is an analog signal, the input interface 111 is provided with an adequate conversion circuit including an A/D converter. This description is similarly applied to other signals that can be received by the input interface 111 and that will be described later.

The information processing device 11 includes a processor 112. The processor 112 is configured to acquire an estimated value of the respiratory rate of the subject 20 based on the waveform signal WF received by the input interface 111.

The information processing device 11 includes an output interface 113. The processor 112 is configured to output, from the output interface 113, a control signal CT that causes the visualizing device 12 to visualize information corresponding to the estimated value of the respiratory rate. The control signal CT may be an analog signal or a digital signal in accordance with the specification of the visualizing device 12.

In other words, the output interface 113 is configured as a hardware interface that can output the control signal CT. In a case where the control signal CT is an analog signal, the output interface 113 provided with an adequate conversion circuit including a D/A converter. This description is similarly applied to other signals that can be outputted from the output interface 113 and that will be described later.

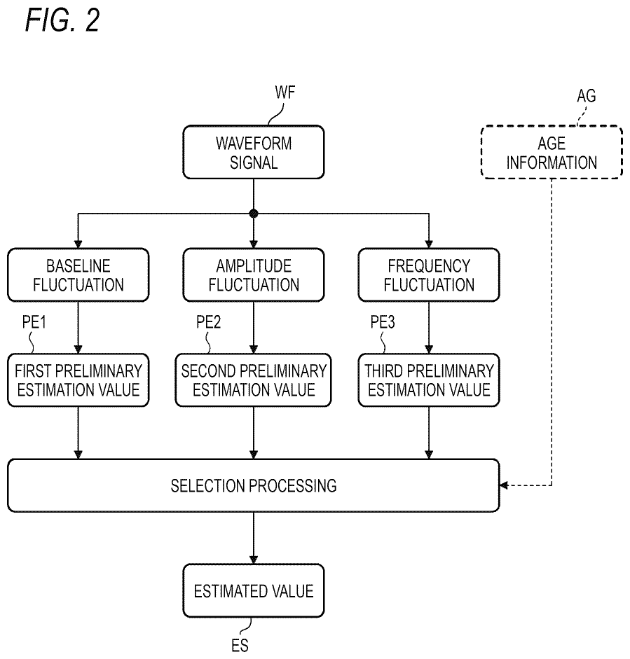

With reference to FIG. 2, a specific flow of processing that is executed by the processor 112 in order to acquire the estimated value of the respiratory rate of the subject 20 will be described.

The waveform of the pulse waves corresponding to the waveform signal WF that is received by the input interface 111 includes a frequency component corresponding to the pulse (0.5 to 2 Hz), a frequency component corresponding to the respiration (0.1 to 1 Hz), and a frequency component of the Mayer wave (0.04 to 0.4 Hz). It is difficult to extract the frequency component corresponding to the respiration only with filtering, because the frequency band thereof partly overlaps with another frequency components.

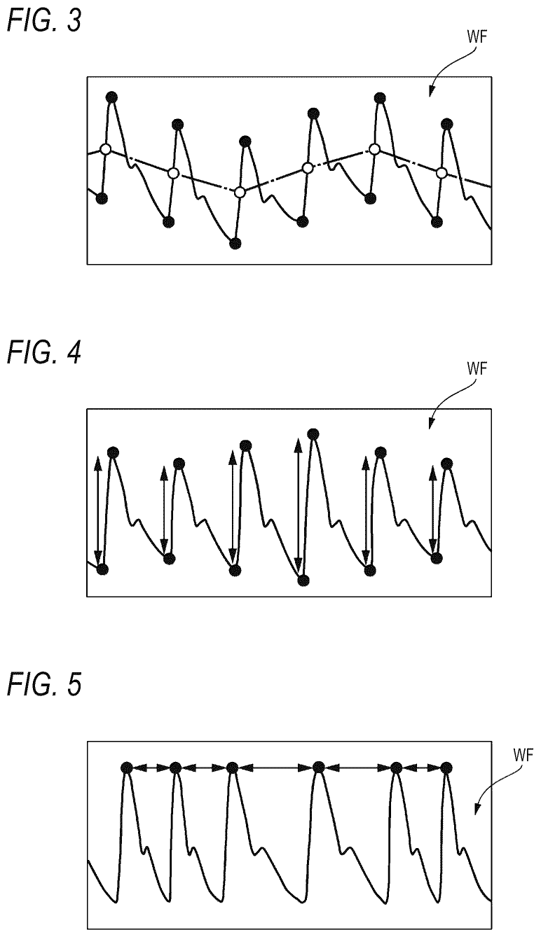

On the other hand, the waveform of the pulse waves includes multiple feature quantities that reflect the influence of respiration. FIG. 3 illustrates a baseline fluctuation of a waveform that is an example of the feature quantity. The baseline fluctuation may be caused by changes in intrathoracic pressure, vasoconstriction of an artery during inspiration, or the like. FIG. 4 illustrates an amplitude fluctuation of a waveform that is another example of the feature quantity. The amplitude fluctuation may be caused by changes in the intrathoracic pressure or the like. FIG. 5 illustrates a frequency fluctuation of a waveform that is another example of the feature quantity. The frequency fluctuation may be caused by a respiratory sinus arrhythmia (RSA), or the like.

Accordingly, as illustrated in FIG. 2, the processor 112 performs processing for extracting the baseline fluctuation, the amplitude fluctuation, and the frequency fluctuation from the waveform signal WF.

Subsequently, the processor 112 performs processing for acquiring a preliminary estimation value of the respiration rate based on each of the multiple extracted feature quantities. Specifically, by performing frequency analysis processing with respect to each of the feature quantities, a frequency having the largest power spectral value is specified. Examples of the frequency analysis processing include a fast Fourier transform, a wavelet transform, and the like. The processor 112 associates the frequency as specified with a preliminary estimation value of the respiratory rate (the number of respirations per minute).

In this example, the estimated value of the respiratory rate that is acquired based on the baseline fluctuation is referred to as a first preliminary estimation value PE1. Similarly, the estimated value of the respiration rate that is acquired based on the amplitude fluctuation is referred to as a second preliminary estimation value PE2, and the estimated value of the respiration rate that is acquired based on the frequency fluctuation is referred to as a third preliminary estimation value PE3.

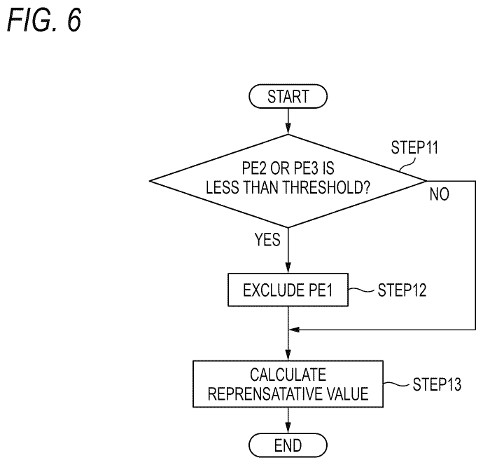

Subsequently, the processor 112 executes a selection processing for selecting at least one of the multiple preliminary estimation values that are acquired as described above, based on state information corresponding to a state of the subject 20. FIG. 6 illustrates an exemplary flow of the selection processing.

In STEP 11, the processor 112 determines whether the second preliminary estimation value PE2 or the third preliminary estimation value PE3 is less than a threshold. In response to the determination that the second preliminary estimation value PE2 or the third preliminary estimation value PE3 is less than the threshold (YES in STEP 11), the processor 112 excludes the first preliminary estimation value PE1 (STEP 12). In other words, the preliminary estimation value of the respiratory rate that is acquired based on the baseline fluctuation is excluded.

Subsequently, the processor 112 calculates a representative value of the second preliminary estimation value PE2 and the third preliminary estimation value PE3 in STEP 13. In this example, the representative value is an average value. The processor 112 handles the calculated average value as a estimated value ES of the respiratory rate of the subject 20 in FIG. 2. In other words, the respiratory rate of the subject is estimated based on the preliminary estimation values acquired based on the amplitude fluctuation and the frequency fluctuation.

In response to the determination that both the second preliminary estimation value PE2 and the third preliminary estimation value PE3 are no less than the threshold (NO in STEP 11), the processor 112 calculates representative values of all the preliminary estimation values (STEP 13). In this example, the representative value is an average value. However, a median value or a mode value may be used as the representative value.

The processor 112 outputs, from the output interface 113, a control signal CT that causes the visualizing device 12 to visualize information corresponding to the calculated estimated value ES of the respiratory rate of the subject 20. The information may be the estimated value itself, or a color, a symbol, a figure, or the like corresponding to the estimated value.

The inventor of the present application paid attention to a fact that the estimation accuracy of the respiration rate based on the baseline fluctuation is deteriorated in a case where a respiration frequency of the subject 20 is low (e.g., 10 or less per minute). According to the configuration of the above-described exemplary processing, the estimated value ES of the respiratory rate of the subject 20 is determined after the first preliminary estimation value PE1 that is acquired based on the baseline fluctuation is excluded, in response to the determination that the respiratory rate that is estimated based on the amplitude fluctuation or the frequency fluctuation of the waveform signal WF corresponding to the waveform of the pulse waves is less than the threshold. Accordingly, the second preliminary estimation value PE2 and the third preliminary estimation value PE3 may be examples of state information corresponding to the state of the subject 20.

Since the processing for excluding the preliminary estimation value that deteriorates the estimation accuracy of the respiration rate based on the respiration frequency of the subject 20 is executed by the processor 112 installed in the information processing device 11, it is possible to suppress the deterioration in the estimation accuracy of the respiration rate in accordance with the respiration frequency of the subject 20 without increasing the number of sensors.

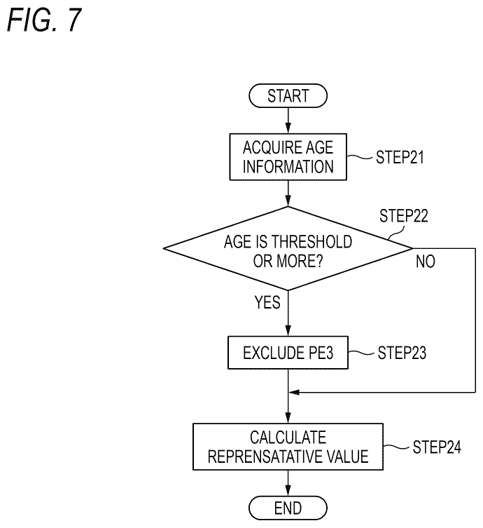

FIG. 7 illustrates another exemplary flow of the selection processing that is executed by the processor 112. In this example, the age information AG of the subject 20 is first acquired (STEP 21). In this example, the age information AG corresponds to a vascular age of the subject 20. The age information AG is an example of the state information.

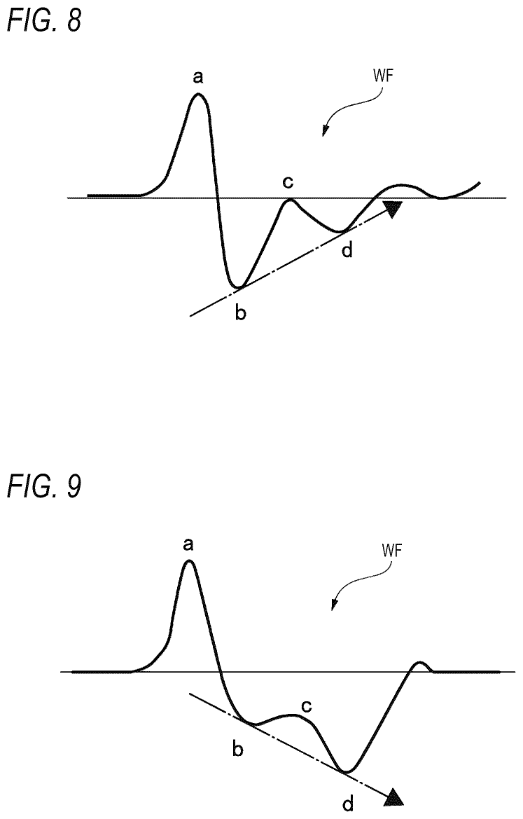

FIG. 8 illustrates a waveform of acceleration pulse waves that is obtained by quadratically differentiating a waveform of the pulse waves that is acquired from a subject whose vascular age is in his/her thirties. FIG. 9 illustrates a waveform of acceleration pulse waves that is similarly acquired from a subject whose vascular age is in his/her seventies. The reference symbol “a” indicates a wave (a-wave) that appears when his/her heart ejects blood. The reference symbol “b” indicates a wave (b-wave) that appears when the a-wave rebounds. The magnitude of the b-wave reflects the flexibility of the blood vessel. The reference symbol “c” indicates a wave (c-wave) whose amplitude is decreasing with aging. The reference symbol “d”indicates a wave (d-wave) reflecting the state of the vessel in the whole body of the subject.

In a case where the vascular age is relatively low, the gradient of a linear line connecting a vertex of the b-wave and a vertex of the d-wave tends to have a positive value. On the other hand, when the vascular age is relatively high, the gradient of the same line tends to have a negative value. Therefore, the vascular age of the subject can be estimated from the value of the gradient of the same line.

For example, the processor 112 may be configured to specify the b-wave and the d-wave from the waveform signal WF received by the input interface 111, and to acquire the value of the gradient of the linear line described above. It is preferable to calculate the value of the gradient as a representative value of multiple gradients that are acquired from multiple pulse waves corresponding to multiple cycles of the pulsation. Examples of the representative value include a mean value, a median value, a mode value, a minimum value, and a maximum value.

Subsequently, in STEP 22, the processor 112 determines whether the age of the subject 20 is no less than a threshold. As an example, the processor 112 is configured to be able to refer to a function in which the value of the gradient is used as an input, and the estimated value of the vascular age is used as an output. In other words, the estimated value of the vascular age may be referred to as the age information AG. In this case, the processor 112 determines whether the age of the subject 20 is no less than the threshold based on whether the estimated value of the vascular age is no less than the threshold.

As another example, the value of the gradient itself may be referred to as the age information AG. For example, in a case where the gradient has a negative value, the age of the subject 20 may be determined to be no less than the threshold.

In response to the determination that the age of the subject 20 is no less than the threshold (YES in STEP 22), the processor 112 excludes the third preliminary estimation value PE3 (STEP 23). In other words, the preliminary estimation value of the respiratory rate that is acquired based on the frequency fluctuation is excluded.

Subsequently, the processor 112 calculates a representative value of the first preliminary estimation value PE1 and the second preliminary estimation value PE2 in STEP 24. In this example, the representative value is an average value. The processor 112 handles the calculated average value as a estimated value ES of the respiratory rate of the subject 20 in FIG. 2. In other words, the respiratory rate of the subject is estimated based on the preliminary estimation values acquired based on the baseline fluctuation and the amplitude fluctuation.

In response to the determination that the age of the subject 20 is less than the threshold (NO in STEP 22), the processor 112 calculates representative values of all the preliminary estimation values (STEP 24). In this example, the representative value is an average value. However, a median value or a mode value may be used as the representative value.

The inventor of the present application paid attention to a fact that the estimation accuracy of the respiration rate based on the frequency fluctuation is deteriorated in a case where the age of the subject 20 is high. According to the configuration of the above-described exemplary processing, the respiratory rate of the subject 20 is finally estimated after the respiratory rate that is estimated based on the frequency fluctuation is excluded, in response to the determination that the age of the subject 20 that is estimated based on the waveform signal WF corresponding to the waveform of the pulse waves is no less than the threshold. Accordingly, the estimated value of the age of the subject 20 may be an example of the state information corresponding to the state of the subject 20.

Since the processing for excluding the preliminary estimation value that deteriorates the estimation accuracy of the respiration rate based on the vascular age of the subject 20 is executed by the processor 112 installed in the information processing device 11, it is possible to suppress the deterioration in the estimation accuracy of the respiration rate in accordance with the vascular age of the subject 20 without increasing the number of sensors.

As illustrated in FIG. 1, the monitoring device 10 may include a user interface 13. The user interface 13 is configured to accept a user's input of age information AG indicating an actual age of the subject 20. The age information AG indicating the actual age may be inputted from a database storing electronic medical record data.

In other words, the age information AG acquired in STEP 21 of the exemplary processing described with reference to FIG. 7 may indicate the actual age of the subject 20. The age information AG indicating the actual age of the subject is the state information corresponding to the state of the subject 20.

According to the above configuration, since the processing for excluding the preliminary estimation value that deteriorates the estimation accuracy of the respiration rate based on the actual age of the subject 20 is performed by the processor 112 installed in the information processing device 11, it is possible to suppress the deterioration in the estimation accuracy of the respiration rate in accordance with the age of the subject 20 without increasing the number of sensors.

The processor 112 of the information processing device 11 having various functions as exemplified above may be implemented by at least one non-exclusive microprocessor configured to cooperate with at least one non-exclusive memory. Examples of the non-exclusive microprocessor include a CPU, an MPU, and a GPU. Examples of the non-exclusive memory include ROM and RAM. In this case, a computer program that implements the various functions described above may be stored in the ROM. The ROM is an example of a non-transitory computer-readable medium having stored a computer program. The non-exclusive microprocessor designates at least a part of the program stored in the ROM, loads the designated program in the RAM, and executes the above-described processing in cooperation with the RAM. The computer program may be pre-installed in a non-exclusive memory, or may be downloaded from an external server device with a communication network, and then installed in the non-exclusive memory. In this case, the external server device is an example of the non-transitory computer-readable medium having stored the computer program.

The processor 112 may be implemented by at least one exclusive integrated circuitry capable of executing the above-described computer program. Examples of the exclusive integrated circuit include a microcontroller, an ASIC, and an FPGA. In this case, the above-described computer program is pre-installed in a memory element included in the dedicated integrated circuit. The memory element is an example of a non-transitory computer-readable medium having stored a computer program. The processor 112 may also be implemented by a combination of the non-exclusive microprocessor and the exclusive integrated circuitry.

The various configurations described above are merely illustrative for facilitating understanding of the presently disclosed subject matter. Each of the illustrative configurations may be appropriately modified or combined with another illustrative configuration within the gist of the present disclosure.

The processing illustrated in FIG. 6 and the processing illustrated in FIG. 7 may be combined. In a case where at least one of the first preliminary estimation value PE1 and the third preliminary estimation value PE3 is excluded based on the waveform signal WF, an estimation value of the respiratory rate of the subject 20 is determined based on the remaining preliminary estimation value including at least the second preliminary estimation value PE2.

In the above exemplary embodiment, the baseline fluctuation, the amplitude fluctuation, and the frequency fluctuation are extracted as the multiple feature quantities related to the waveform signal WF. However, a configuration in which two of the feature quantities are extracted may also be employed. In this case, it is preferable that one of the two feature quantities is the amplitude fluctuation.

According to the above configuration, since the processing for excluding the preliminary estimation value that deteriorates the estimation accuracy of the respiration rate based on the state of the subject 20 is executed by the processor 112 installed in the information processing device 11, it is possible to suppress the deterioration in the estimation accuracy of the respiration rate in accordance with the state of the subject 20 without increasing the number of sensors.

In the above exemplary embodiment, the estimated value ES of the respiratory rate of the subject 20 is visualized by the visualizing device 12. However, the estimated value ES may be outputted as sound by a speaker, or may be transmitted to another device as data by a data transmission device. In this case, the speaker or the data transmission device may be an example of the output device.

In the above exemplary embodiment, a waveform signal WF corresponding to the waveform of the pulse waves of the subject 20 is acquired. However, a waveform signal WF corresponding to a waveform of the electrocardiogram of the subject 20 may be acquired. In other words, the electrocardiogram may be an example of physiological information involving cyclic changes.

1. An information processing device configured to process physiological information of a subject, comprising:
an interface configured to receive a signal corresponding to a waveform of physiological information involving cyclic changes from a sensor attached to the subject; and
a processor configured to cause, in response to the signal, an output device to output information corresponding to an estimated value of a respiration rate of the subject,
wherein the processor is configured to:
extract multiple feature quantities corresponding to respiration of the subject from the signal;
apply frequency analysis processing with respect to each of the feature quantities to acquire multiple preliminary estimation values of the respiration rate;
exclude one of the preliminary estimation values in response to state information corresponding to a state of the subject; and
determine the estimated value based on at least one of the preliminary estimation values as remained.

2. The information processing device according to claim 1,
wherein the feature quantities include a base line fluctuation, an amplitude fluctuation, and a frequency fluctuation;
wherein the state information is an estimated value of a respiration rate that is acquired based on the amplitude fluctuation or the frequency fluctuation; and
wherein one of the preliminary estimation values that is acquired based on the baseline fluctuation is excluded.

3. The information processing device according to claim 1,
wherein the feature quantities include a frequency fluctuation;
wherein the state information corresponds to an age of the subject; and
wherein one of the preliminary estimation values that is acquired based on the frequency fluctuation is excluded.

4. The information processing device according to claim 3,
wherein the state information corresponds to a vascular age of the subject that is acquired based on the signal.

5. The information processing device according to claim 1,
wherein the physiological information includes pulse waves.

6. A monitoring device configured to monitor physiological information of a subject, comprising:
an interface configured to receive a signal corresponding to a waveform of physiological information involving cyclic changes from a sensor attached to the subject;
an output device; and
a processor configured to cause, in response to the signal, the output device to output information corresponding to an estimated value of a respiration rate of the subject,
wherein the processor is configured to:
extract multiple feature quantities corresponding to respiration of the subject from the signal;
apply frequency analysis processing with respect to each of the feature quantities to acquire multiple preliminary estimation values of the respiration rate;
exclude one of the preliminary estimation values in response to state information corresponding to a state of the subject; and
determine the estimated value based on at least one of the preliminary estimation values as remained.

7. A non-transitory computer-readable medium having stored a computer program adapted to be executed by a processor installed in an information processing device configured to process physiological information of a subject, the computer program being configured to, when executed, cause the information processing device to:
receive a signal corresponding to a waveform of physiological information involving cyclic changes from a sensor attached to the subject;
extract multiple feature quantities corresponding to respiration of the subject from the signal;
apply frequency analysis processing with respect to each of the feature quantities to acquire multiple preliminary estimation values of a respiration rate of the subject;
exclude one of the preliminary estimation values in response to state information corresponding to a state of the subject;
determine an estimated value of the respiration rate based on at least one of the preliminary estimation values as remained; and
cause an output device to output information corresponding to the estimated value.
